(19) United States
(12) Patent Application Publication
(10) Pub. No.: US 2026/0092921 A1
(43) Pub. Date: Apr. 2, 2026

(54) MUTATED NEUROAMINIDASE

(71) Applicants: Roche Diagnostics GmbH, Mannheim (DE); Ventana Medical Systems, Inc., Tucson, AZ (US)

(72) Inventors: Mara Boenitz-Dulat, Tutzing (DE); Rui Hong, Oro Valley, AZ (US); Anna Nolte, Penzberg (DE); Rodrigo Velasco Bucheli, Bad Heilbrunn (DE)

(21) Appl. No.: 19/336,417

(22) Filed: Sep. 22, 2025

Related U.S. Application Data
(60) Provisional application No. 63/699,948, filed on Sep. 27, 2024.

(30) Foreign Application Priority Data
Oct. 10, 2024 (EP) 24205944.2

(51) Int. Cl. G01N 33/573; C12N 9/24

(52) U.S. Cl. CPC G01N 33/573; C12N 9/2402; C12Y 302/01018

(57) ABSTRACT

The present invention relates to a mutated neuraminidase enzyme, or a functional fragment thereof with increased enzymatic activity at alkaline pH, compared to a non-mutated neuraminidase enzyme. The invention further provides nucleic acids encoding the mutant enzyme, a vector, a recombinant cell comprising the nucleic acid and a method for producing the enzyme. Also provided are a method for immunohistochemical (IHC) staining, the use of the mutated enzyme in immunohistochemical (IHC) staining, as well as a kit comprising the mutated enzyme.

### CROSS-REFERENCE TO RELATED APPLICATIONS

This application claims priority to U.S. Patent Application No. 63/699,948 filed on Sep. 27, 2024 and European Patent Application No. 24205944.2 filed on Oct. 10, 2024, the contents of each application are incorporated herein by reference in their entireties.

### SEQUENCE LISTING

This application incorporates by reference in its entirety the material in the ST.26 XML file titled R31237US.xml, which was created on Sep. 22, 2025 and is 10,698 bytes.

The present invention relates to a mutated neuraminidase enzyme or a functional fragment thereof having increased enzymatic activity at alkaline pH compared to a non-mutated neuraminidase enzyme. The invention further provides nucleic acids encoding the mutant enzyme, a vector, a recombinant cell comprising the nucleic acid and a method for producing the enzyme. Also provided are a method for immunohistochemical (IHC) staining, the use of the mutated enzyme in immunohistochemical (IHC) staining, as well as a kit comprising the mutated enzyme.

### DESCRIPTION

Immunohistochemistry is an extensively used technique, whereby antibodies are used to detect antigens in cells and/or a tissue section. Albert Hewett Coons, Hugh J Creech, Norman Jones, and Ernst Berliner conceptualized and first implemented the procedure of immunofluorescence in 1941, which led to the later development of immunohistochemistry (1). They used fluorescein isothiocyanate (FITC)-labelled antibodies to localize pneumococcal antigens in infected tissues. Since then, with the development of protein conjugation, enzyme labels have been introduced, such as peroxidase and alkaline phosphatase. The history of immunohistochemistry (IHC) combines physiology, immunology, biochemistry, and the work of various Nobel Prize laureates. From von Behring who was awarded the first Nobel Prize in 1901 for his work on serum therapy to the 1984 Nobel Prize for the discovery of monoclonal antibodies by Milstein, Kohler, and Jerne.

Immunohistochemical staining has numerous applications in the diagnosis of abnormal cells such as those found in cancerous tumours. In some cancer cells certain tumour antigens are expressed making it possible to detect them. Immunohistochemistry is also widely used in basic research, to understand the distribution and localization of biomarkers and differentially expressed proteins in different parts of a biological tissue (2).

Furthermore, immunohistochemistry can be performed on tissue that has been fixed and embedded in paraffin, but also on cryopreserved tissue. Based on the way the tissue is preserved, there are different steps to prepare the tissue for immunohistochemistry, but the general method includes proper fixation and antigen detection via direct or indirect detection methods (3).

The so-called direct method is a one-step staining method and involves an enzyme labelled antibody reacting directly with the antigen in tissue sections. While this technique utilizes only one antibody and consequently is simple and rapid, the sensitivity is lower due to little signal amplification, in contrast to indirect detection methods (4).

The indirect method uses an unlabelled primary antibody that binds to the target antigen in the tissue. Then a secondary antibody is added as a second layer, which binds with the primary antibody. The secondary antibody is raised against the antibody IgG of the animal species in which the primary antibody has been raised. This method is more sensitive than direct detection strategies because of signal amplification due to the binding of several secondary antibodies to each primary antibody (4).

The reporter molecules in immunohistochemistry vary based on the nature of the detection method. The most common reporter molecules are chromogenic and fluorescence detection. In chromogenic immunohistochemistry an antibody is conjugated to an enzyme, such as alkaline phosphatase and horseradish peroxidase, that can catalyse a colour-producing reaction in the presence of a chromogenic substrate like diaminobenzidine (5). The coloured product can be analysed with an ordinary light microscope (6).

In immunofluorescence, an antibody is conjugated to a fluorophore, such as fluorescein isothiocyanate, tetramethylrhodamine isothiocyanate, aminomethyl Coumarin acetate or Cyanine 5. The fluorochromes can be visualized by a fluorescence or confocal microscope (6).

The most common enzymes in immunohistochemistry are alkaline phosphatase and horseradish peroxidase. The most common buffer is a Tris-EDTA buffer with a pH of 9. The relatively small selection of established enzymes and the preferred alkaline pH open up a need for new possible enzymes for immunohistochemistry, which are as active as the established enzymes in an alkaline pH range.

Neuraminidases (NEUs), also known as sialidases, are a family of enzymes that cleave sialic acid on the surfaces of cells. The functions of viral NEUs have been well documented in the influenza virus replication. The biological effects of mammalian NEUs, however, tend to be underestimated and are less characterized. Mammalian NEUs catalyse the cleavage of terminal sialic acids from glycoproteins or glycolipids. Four types of mammalian NEUs (NEU1, NEU2, NEU3, and NEU4) have been identified to date, differing in their subcellular localization and enzymatic properties. Of these, NEU1 is the most abundantly expressed. In addition to its typical catabolic function in lysosomes, NEU1 can also translocate to the cell surface, where it is involved in structural and functional modulation of cellular receptors (7).

Some examples of receptors and cellular signalling modulated by NEU1 desialylation at the cell membrane include TLR4-NFκB-associated response, insulin receptor-related glucose uptake, Fc receptors for immunoglobulin G in macrophage phagocytosis, and epidermal growth factor receptor and insulin-like growth factor-2 in proliferation (8).

WO 2024/131237 discloses a recombinant viral vector for treating sialidosis. Sialidosis is a rare lysosomal storage disease caused by the mutation of the neuraminidase 1 (NEU1) gene that leads to a deficiency of neuraminidase 1 (also known as “sialidase”) in the affected subjects. This disclosure aims at providing a recombinant adeno-associated virus (rAAV) for treating sialidosis. The recombinant AAV of this invention is characterized by carrying two therapeutic genes, NEU1 and CTSA, in its viral genome.

U.S. Pat. No. 10,525,109 B2 discloses that influenza viruses belong to the orthomyxoviridae family of RNA viruses. Both type A and type B viruses have 8 segmented negative-strand RNA genomes enclosed in a lipid envelope derived from the host cell. The viral envelope is covered with spikes that are composed of three proteins: hemagglutinin (HA), that attaches virus to host cell receptors and mediates fusion of viral and cellular membranes; neuraminidase (NA), which facilitates the release of the new viruses from the host cell, and a small number of M2 proteins that serve as ion channels. Therefore, neuraminidase are targets for preventing pathogen infection.

US 2020/0087643 A1 relates to enzymes and combinations thereof useful for studying glycoproteins, and corresponding methods of use. In particular, the invention relates to a sialidase composition comprising an additional protease and/or glycosidase, preferably an 0-glycoprotein-specific endoprotease and/or an O-glycosidase.

Despite all above research on neuraminidases, no information is provided about the use of neuraminidases in immunohistochemistry.

Because of the relatively small selection of established enzymes in immunohistochemistry with high enzymatic activity at an alkaline pH, there is a need for new enzymes for immunohistochemistry that still show comparable activities to the established enzymes in an alkaline pH range. Other objects and advantages of the present invention will become apparent to the person skilled in the art when studying the following more detailed description of the present invention, including the Figures and examples.

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

The present invention solves the above object providing a recombinantly expressed neuraminidase with optimized pH activity and stability in the alkaline range (pH 9-10) while at the same time maintaining sufficient optimizing enzymatic performance (kinetic parameters).

In the first aspect thereof, the invention relates to a recombinant mutated neuraminidase enzyme or a functional fragment thereof, comprising an amino acid sequence having at least 85%, more preferably at least 90% sequence identity to SEQ ID NO. 4, further comprising at least one mutation between residues 115 and 680 of SEQ ID NO: 4, wherein said recombinant mutated neuraminidase enzyme or functional fragment thereof exhibits an increased enzymatic activity at alkaline pH when compared to the non-mutated neuraminidase enzyme.

In preferred embodiments, the mutated neuraminidase of the invention is an isolated neuraminidase or a recombinant neuraminidase polypeptide. The term “recombinant” or “recombinantly produced” in context of the invention shall mean that a protein or peptide is expressed via an artificially introduced exogenous nucleic acid sequence in a biological cell. Recombinant expression is usually performed by using expression vectors as described herein.

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

As used herein, the terms “identical” or percent “identity”, when used anywhere herein in the context of two or more nucleic acid or protein/polypeptide sequences, refer to two or more sequences or subsequences that are the same or have (or have at least) a specified percentage of amino acid residues or nucleotides that are the same (i.e., at, or at least, about 60% identity, preferably at, or at least, 65%, 70%, 75%, 80%, 85%, 90%, 91%, 92%, 93% or 94%, identity, and more preferably at, or at least, about 95%, 96%, 97%, 98%, 99%, or higher identity over a specified region—preferably over their full length sequences—, when compared and aligned for maximum correspondence over the comparison window or designated region) as measured using a sequence comparison algorithms, or by manual alignment and visual inspection (see, e.g., NCBI web site).

In a particular embodiment, for example when comparing the protein or nucleic acid sequence of a mutated neuraminidase with a wild-type neuraminidase, the percentage identity can be determined by the Blast searches or local alignments; in particular for amino acid identity, those using BLASTP 2.2.28+ with the following parameters: Matrix: BLOSUM62; Gap Penalties: Existence: 11, Extension: 1; Neighbouring words threshold: 11; Window for multiple hits: 40.

In view of the results as obtained in the experiments as performed in the context of the present invention, the inventors found that six neuraminidases that contain disulfide bridges may provide the required activity. The six candidates were exo-alpha-sialidase NanI from Clostridium perfringens (NCBI Reference Sequence: WP_011590331.1), LPXTG-anchored neuraminidase NanA from Streptococcus pneumoniae (NCBI Reference Sequence: WP_014632427.1), extracellular sialidase/neuraminidase, putative Af293 from Aspergillus fumigatus (GenBank: EAL89414.2), sialidase family protein AkNeu from Halalkalibacter krulwichiae (NCBI Reference Sequence: WP_084372094.1), hypothetical protein AHA02nite_24160 AhNeu from Alkalibacillus haloalkaliphilus (GenBank: GEN46640.1), and sialidase family protein AsNeu from Shouchella shacheensis (NCBI Reference Sequence: WP_059104496.1).

After additional experiments, the candidate AfNeu was excluded because of the low acceptance of substrates. AsNeu and NanI are regarded less preferred after determining their optimum pH conditions at pH 7.0 to 7.4.

The rest of the experiments were performed with NanA, AkNeu and AhNeu. It became evident after comparing the initial velocity, time to maximum velocity (time to plateau) and pH optimum, that AkNeu constituted the best enzyme as a starting point for further optimization as described herein.

Preferred is the enzyme or the functional fragment thereof according to the invention, further comprising at least one additional mutation, preferably two mutations between residues 400 and 420 of SEQ ID NO: 4, wherein said recombinant mutated neuraminidase enzyme or functional fragment thereof further exhibits a higher/increased kinetic activity at alkaline pH when compared to the non-mutated neuraminidase enzyme.

The term “enzymatic activity” in this invention generally refers to the relative activity of a mutated neuraminidase enzyme or functional fragment thereof in alkaline pH higher than pH 8.0 in comparison to the enzymatic activity of the non-mutated neuraminidase enzyme or functional fragment thereof at pH 8.0 and 33° C.

The term “kinetic activity” in this invention refers to kinetic parameters in respect to non-mutated neuraminidase enzyme or functional fragment thereof. The parameters are measured in kcat, KM, and kcat/KM and are known to a person skilled in the art.

Preferred is the enzyme or the functional fragment thereof according to the invention, wherein the neuraminidase enzyme is derived from the sialidase family protein of the bacterium Halalkalibacter krulwichiae, preferably according to SEQ ID NO: 4, or a bacterial homolog thereof having neuraminidase activity.

The term “homolog” refers to homologous sequences having the same, or substantially the same enzymatic functions. Homology is inferred based on sequence similarity throughout the different living beings. All methods known to a person skilled in the art can be used to identify sequences that have statistically significant similarity.

Sialidases or neuraminidases function to bind and hydrolyze terminal sialic acid residues from various glycoconjugates, they play vital roles in pathogenesis, bacterial nutrition and cellular interactions. They have a six-bladed, beta-propeller fold with the non-viral sialidases containing 2-5 Asp-box motifs (most commonly Ser/Thr-X-Asp-[X]-Gly-X-Thr-Trp/Phe). This common domain includes eubacterial and eukaryotic sialidases.

Based on a structural alignment, the following residues are likely to form the AkNeu substrate-binding site: R251, R270, D276, D313, Y476, Q484, E530, R546, R607, Y647, and E663.

Therefore, further preferred is the enzyme or a functional fragment thereof according to the invention The enzyme or the functional fragment thereof according to the invention, wherein the position of the at least one mutation is selected from the group consisting of positions 115, 221, 228, 231, 405, 412, and 679 according to SEQ ID NO: 4.

A preferred mutated neuraminidase of the invention is a protein having at least 65%, 70%, 75%, 80%, 85%, 86%, 87%, 88%, or 89% identity, and more preferably at, or at least, about 90%, 91%, 92%, 93% or 94%, even more preferably 95%, 96%, 97%, 98%, 99%, or 100% sequence identity compared to the sequence shown in SEQ ID NO: 4. Also included are functional fragments of these proteins that retain or substantially retain the neuraminidase catalytic activity.

Further preferred is the enzyme or a functional fragment thereof according to the invention, wherein the mutation is selected from a substitution, deletion, or amino acid modification, and preferably is an amino acid substitution.

The term “mutation” refers to, in the context of a polynucleotide, a modification to the poly-nucleotide sequence resulting in a change in the sequence of a polynucleotide with reference to a precursor polynucleotide sequence. A mutant polynucleotide sequence can refer to an alteration that does not change the encoded amino acid sequence, for example, with regard to codon optimization for expression purposes, or that modifies a codon in such a way as to result in a modification of the encoded amino acid sequence. Mutations can be introduced into a polynucleotide through any number of methods known to those of ordinary skill in the art, including random mutagenesis, site-specific mutagenesis, oligonucleotide directed mutagenesis, gene shuffling, directed evolution techniques, combinatorial mutagenesis, site saturation mutagenesis among others.

“Mutation” or “mutated” means, in the context of a protein, a modification to the amino acid sequence resulting in a change in the sequence of a protein with reference to a precursor protein sequence. A mutation can refer to a substitution of one amino acid with another amino acid, an insertion or a deletion of one or more amino acid residues. Specifically, a mutation can also be the replacement of an amino acid with a non-natural amino acid, or with a chemically modified amino acid or like residues. A mutation can also be a truncation (e.g., a deletion or interruption) in a sequence or a subsequence from the precursor sequence. A mutation may also be an addition of a subsequence (e.g., two or more amino acids in a stretch, which are inserted between two contiguous amino acids in a precursor protein sequence) within a protein, or at either terminal end of a protein, thereby increasing the length of (or elongating) the protein. A mutation can be made by modifying the DNA sequence corresponding to the precursor protein. Mutations can be introduced into a protein sequence by known methods in the art, for example, by creating synthetic DNA sequences that encode the mutation with reference to precursor proteins, or chemically altering the protein itself. A “mutant” as used herein is a protein comprising a mutation. For example, it is also possible to make a mutant by replacing a portion of neuraminidase with a wild-type sequence that corresponds to such portion but includes a desired variation at a specific position that is naturally occurring in the wild-type sequence.

Also, preferred is the enzyme or the functional fragment thereof according to the invention, wherein the at least one mutation is selected from the group consisting of positions D115N, E221R, P228A, A231C, D405C, T412C, and F679C of SEQ ID NO. 4.

Further preferred is the enzyme or the functional fragment thereof according to the invention, wherein the amino acid sequence of the recombinant mutated neuraminidase enzyme or the functional fragment thereof comprises two mutations selected from the group consisting of A231C and F679C, and D405C and T412C.

Therefore, also provided in some embodiments is a functional fragment of a mutated neuraminidase of the invention. The functional fragment preferably comprises, or consists of, or consists essentially of 50 amino acids, preferably 80, more preferably at least 100, more preferably 200, 300 or 400 or 450 amino acids, under the provision that said functional fragment of a mutated neuraminidase retains the neuraminidase catalytic activity as described herein, and preferably comprises a double mutation selected from A231C and F679C or D405C and T412C compared to SEQ ID NO: 4.

Also preferred is the enzyme or the functional fragment thereof according to the invention, wherein the recombinant mutated neuraminidase enzyme, or the functional fragment thereof exhibits an increased enzymatic activity at a pH range of between 8.0 to 10, preferably of between 8.5 to 9.5, and more preferably of between 9.0 to 9.5 compared to the non-mutated neuraminidase enzyme.

The use of the mutated neuraminidase enzymes, or of the functional fragment thereof, of the present invention overcomes the problems in the art because their increased enzymatic activity in an alkaline pH allows the use of the recombinant mutated neuraminidase enzyme or a functional fragment thereof for immunohistochemical (IHC) staining.

Further preferred is the enzyme or the functional fragment thereof according to the invention, wherein the recombinant mutated neuraminidase enzyme, or the functional fragment thereof hydrolases and catalyses the hydrolysis of N-acetylneuraminic acid (or sialic acid) from an adjacent glycoprotein, glycolipids and polysaccharides as well as wild-type neuraminidase enzyme (SEQ ID NO: 4).

In a second aspect the problem is solved by a nucleic acid encoding the enzyme as described herein before, or for the functional fragment of a mutated neuraminidase enzyme as described herein before.

The term “encoding” or more simply “coding” refers to the ability of a nucleotide sequence to code for one or more amino acids. The term does not require a start or stop codon. An amino acid sequence can be encoded in any one of six different reading frames provided by a polynucleotide sequence and its complement. An amino acid sequence can be encoded by desoxyribonucleic acid (DNA), ribonucleic acid (RNA), or artificially synthesized polymers similar to DNA or RNA.

Another aspect of the invention is a vector, comprising the nucleic acid according to the invention.

Preferred is the vector according to the invention, which is an expression vector, preferably comprising a promoter sequence operably linked to the nucleic acid according to invention.

A “vector” may be any agent that is able to deliver or maintain a nucleic acid in a host cell and includes, for example, but is not limited to, plasmids (e.g., DNA plasmids), naked nucleic acids, viral vectors, viruses, nucleic acids complexed with one or more polypeptide or other molecules, as well as nucleic acids immobilized onto solid phase particles. Vectors are described in detail below. A vector can be useful as an agent for delivering or maintaining an exogenous gene and/or protein in a host cell. A vector may be capable of transducing, transfecting, or transforming a cell, thereby causing the cell to replicate or express nucleic acids and/or proteins other than those native to the cell or in a manner not native to the cell. The target cell may be a cell maintained under cell culture conditions or in other in vivo embodiments, being part of a living organism. A vector may include materials to aid in achieving entry of a nucleic acid into the cell, such as a viral particle, liposome, protein coating, or the like. Any method of transferring a nucleic acid into the cell may be used; unless otherwise indicated, the term vector does not imply any particular method of delivering a nucleic acid into a cell or imply that any particular cell type is the subject of transduction. The present invention is not limited to any specific vector for delivery or maintenance of any nucleic acid of the invention, including, e.g., a nucleic acid encoding a mutant neuraminidase polypeptide of the invention or a fragment thereof.

Preferably the vector of the invention is an expression vector. The term “expression vector” typically refers to a nucleic acid construct or sequence, generated recombinantly or synthetically, with a series of specific nucleic acid elements that permit transcription of a particular nucleic acid in a host cell. The expression vector typically includes a nucleic acid to be transcribed—the mutated neuraminidase of the invention—operably linked to a promoter. The term “expression” includes any step involved in the production of the polypeptide including, but not limited to, transcription, post-transcriptional modification, translation, post-translational modification, and/or secretion. A preferred vector of the invention is a plant-specific, bacterial, yeast, insect, vertebrate, preferably mammalian, or a viral vector, preferably retroviral and adeno-associated viral vector. Preferred vectors of the invention are suitable for use in gene therapy, preferably gene therapy based on transformation of autologous adult stem cells.

Another aspect of the invention is a recombinant cell, comprising the enzyme or a functional fragment thereof according to the invention, a nucleic acid according to the invention, or a vector according to the invention.

A “recombinant cell” or also referred to as “host cell” is any cell that is susceptible to transformation and has been transformed with a nucleic acid. Preferably the recombinant or host cell of the invention is a plant cell, bacterial cell, yeast cell, an insect cell or a vertebrate, preferably a mammalian, cell. A preferred recombinant cell is selected from a cell suitable for recombinant expression of the mutated neuraminidase of the invention.

In some embodiments, the mutated neuraminidase enzymes, or the functional fragment thereof, suitable for the present invention are produced in mammalian cells. Non-limiting examples of mammalian cells that may be used in accordance with the present invention include BALB/c mouse myeloma line (NSO/1, ECACC No:85110503); human retinoblasts (PER.C6, CruCell, Leiden, The Netherlands); monkey kidney CV1 line transformed by SV40 (COS-7, ATCC CRL 1651); human embryonic kidney line (293 or 293 cells subcloned for growth in suspension culture, Graham et al, J. Gen Virol, 36:59,1977); human fibrosarcoma cell line (e.g., HT1080); baby hamster kidney cells (BHK, ATCC CCL 10); Chinese hamster ovary cells+/−DHFR (CHO, Urlaub and Chasm, Proc. Natl. Acad. Sci. USA, 77:4216, 1980); mouse Sertoli cells (TM4, Mather, Biol. Reprod., 23:243-251, 1980); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3 A, ATCC CRL 1442); human lung cells (W138, ATCC CCL 75); human liver cells (Hep G2, HB 8065); mouse mammary tumor (MMT 060562, ATCC CCL 51); TRI cells (Mather et al, Annals N.Y. Acad. Sci., 383:44-68, 1982); MRC 5 cells; FS4 cells; and a human hepatoma line (Hep G2).

In some embodiments, the mutated neuraminidase enzymes, or the functional fragment thereof, suitable for the present invention are produced in bacterial cells. Non-limiting examples of bacterial cells that may be used in accordance with the present invention include Escherichia coli (E. coli), preferably E. coli BL21.

Another aspect of the invention is a method for producing the enzyme or the functional fragment thereof according to the invention, comprising the steps of: i) Culturing and harvesting recombinant cells according to the invention expressing the recombinant enzyme, and ii) suitably isolating the enzyme as expressed.

In some embodiments, the mutated neuraminidase enzymes or the functional fragment thereof as suitable for the present invention are produced in cell-free in vitro translation systems. Non-limiting examples of cell-free in vitro translation systems that may be used in accordance with the present invention include cell-free translation systems consisting of extracts from rabbit reticulocytes, wheat germ and Escherichia coli. All are prepared as crude extracts containing all the macromolecular components (70S or 80S ribosomes, tRNAs, aminoacyl-tRNA synthetases, initiation, elongation and termination factors, etc.) required for translation of exogenous RNA. To ensure efficient translation, each extract must be supplemented with amino acids, energy sources (ATP, GTP), energy regenerating systems (creatine phosphate and creatine phosphokinase for eukaryotic systems, and phosphoenol pyruvate and pyruvate kinase for the E. coli lysate), and other co-factors (Mg²⁺, K⁺, etc.). In a further embodiment, the mutated neuraminidase enzymes, or the functional fragment thereof are produced in cell-free in vitro transcription translation systems. These systems for example consist of extracts from E. coli. In these systems DNA is used as a template and during transcription, the 5′ end of the RNA becomes available for ribosomal binding and undergoes translation while its 3′ end is still being transcribed.

Another aspect of the invention is a method for immunohistochemical (IHC) staining of a biological target, such as a cell or tissue section, comprising the steps of i) suitably coupling or complexing the recombinant mutated neuraminidase enzyme or a functional fragment thereof according to the present invention to a suitable binder, such as an antibody or antigen binding fragment thereof, to produce a tagged binder ii) contacting the tagged binder with a sample suspected to contain the biological target together with a suitable chromogenic substrate for the enzyme, such as 5-Carboxytetramethylrhodamin (5-TAMRA), Cyanine5 (Cy5), Dibenzocyclooctyne-Cy5 (DBCO-Cy5), and 4-(4-Dimethylaminophenylazo)-benzolsulfonylchlorid (Dabsylchlorid) at an alkaline pH, iii) immunohistochemical (IHC) staining of the biological target, and optionally iv) detecting the immunohistochemical (IHC) staining.

Preferred is the use of a recombinant mutated neuraminidase enzyme, or a functional fragment thereof in an immunohistochemical (IHC) staining, wherein the enzyme or a functional fragment thereof according to invention is coupled to an antibody for the detection of a specific antigen as a biological target, and mixed with a substrate, preferably a chromogenic substrate active at alkaline pH, such as 5-Carboxytetramethylrhodamin (5-TAMRA), Cyanine5 (Cy5), Dibenzocyclooctyne-Cy5 (DBCO-Cy5), and 4-(4-Dimethylaminophenylazo)-benzolsulfonylchlorid (Dabsylchlorid).

The coupled antibody can be the only antibody in the system for a direct method in a one-step staining method or the coupled antibody can be a secondary antibody that has the ability to bind to a primary antibody bound to a target antigen.

Another aspect of the invention is a kit for performing an immunohistochemical (IHC) staining, comprising the enzyme or a functional fragment thereof according to the present invention, optionally coupled to an antibody or fragment thereof, a suitable substrate for the enzyme or fragment thereof, preferably a chromogenic substrate, and additional materials for immunohistochemical (IHC) staining such as, for example, buffers and instructions for use.

Another aspect of the invention is the use of the recombinant mutated neuraminidase enzyme or the functional fragment thereof according to the invention or the kit according to the invention for immunohistochemical (IHC) staining according to the invention.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

In view of the above, it will be appreciated that the present invention also relates to the following itemised embodiments:

Item 1. A recombinant mutated neuraminidase enzyme or a functional fragment thereof, comprising an amino acid sequence having at least 85%, more preferably at least 90% sequence identity to SEQ ID NO. 4, further comprising at least one mutation between residues 115 and 680 of SEQ ID NO: 4, wherein said recombinant mutated neuraminidase enzyme or functional fragment thereof exhibits an increased enzymatic activity at alkaline pH when compared to the non-mutated neuraminidase enzyme.

Item 2. The enzyme or the functional fragment thereof according to Item 1, further comprising at least one additional mutation, preferably two mutations between residues 400 and 420 of SEQ ID NO: 4, wherein said recombinant mutated neuraminidase enzyme or functional fragment thereof further exhibits an increased kinetic activity at alkaline pH when compared to the non-mutated neuraminidase enzyme.

Item 3. The enzyme or the functional fragment thereof according to Item 1 or 2, wherein the neuraminidase enzyme is derived from the sialidase family protein of the bacterium Halalkalibacter krulwichiae, preferably according to SEQ ID NO: 4, or a bacterial homolog thereof having neuraminidase activity.

Item 4. The enzyme or the functional fragment thereof according to any one of Items 1 to 3, wherein the position of the at least one mutation is selected from the group consisting of positions 115, 221, 228, 231, 405, 412, and 679 according to SEQ ID NO: 4.

Item 5. The enzyme or the functional fragment thereof according to any one of Items 1 to 4, wherein the mutation is selected from a substitution, deletion, or amino acid modification, and preferably is an amino acid substitution.

Item 6. The enzyme or the functional fragment thereof according to any one of Items 1 to 5, wherein the at least one mutation is selected from the group consisting of D115N, E221R, P228A, A231C, D405C, T412C, and F679C.

Item 7. The enzyme or the functional fragment thereof according to any one of Items 1 to 6, wherein the amino acid sequence of the recombinant mutated neuraminidase enzyme or the functional fragment thereof comprises two mutations selected from the group consisting of A231C and F679C, and D405C and T412C.

Item 8. The enzyme or the functional fragment thereof according to any one of Items 1 to 7, wherein the recombinant mutated neuraminidase enzyme, or the functional fragment thereof exhibits an increased enzymatic activity at a pH range of between 8.0 to 10, preferably of between 8.5 to 9.5, and more preferably of between 9.0 to 9.5 compared to the non-mutated neuraminidase enzyme.

Item 9. A nucleic acid encoding the enzyme or the functional fragment thereof according to any one of Items 1 to 8.

Item 10. A vector, comprising the nucleic acid according to Item 9.

Item 11. The vector according to Item 10, which is an expression vector, preferably comprising a promoter sequence operably linked to the nucleic acid according to Item 9.

Item 12. A recombinant cell, comprising the enzyme or the functional fragment thereof according to any one of Items 1 to 8, the nucleic acid according to Item 9, or the vector according to Item 10 or 11.

Item 13. A method for producing the enzyme or the functional fragment thereof according to any one of Items 1 to 8, comprising the steps of: i) Culturing and harvesting recombinant cells according to Item 12 expressing the recombinant enzyme, and ii) suitably isolating the enzyme as expressed.

Item 14. A method for immunohistochemical (IHC) staining of a biological target, such as a cell or tissue section, comprising the steps of i) suitably coupling or complexing the recombinant mutated neuraminidase enzyme or a functional fragment thereof according to any one of Items 1 to 8 to a suitable binder, such as an antibody or antigen binding fragment thereof, to produce a tagged binder ii) contacting the tagged binder with a sample suspected to contain the biological target together with a suitable chromogenic substrate for the enzyme, such as 5-Carboxytetramethylrhodamin (5-TAMRA), Cyanine5 (Cy5), Dibenzocyclooctyne-Cy5 (DBCO-Cy5), and 4-(4-Dimethylaminophenylazo)-benzolsulfonylchlorid (Dabsylchlorid) at an alkaline pH, iii) immunohistochemical (IHC) staining of the biological target, and optionally iv) detecting the immunohistochemical (IHC) staining.

Item 15. A kit for performing an immunohistochemical (IHC) staining, comprising the enzyme or a functional fragment thereof according to any one of Items 1 to 8, optionally coupled to an antibody or fragment thereof, a suitable substrate for the enzyme or fragment thereof, preferably a chromogenic substrate, and additional materials for immunohistochemical (IHC) staining such as, for example, buffers and instructions for use.

Item 16. Use of the recombinant mutated neuraminidase enzyme or the functional fragment thereof according to any one of Items 1 to 8 or the kit according to Item 15 for immunohistochemical (IHC) staining according to Item 14.

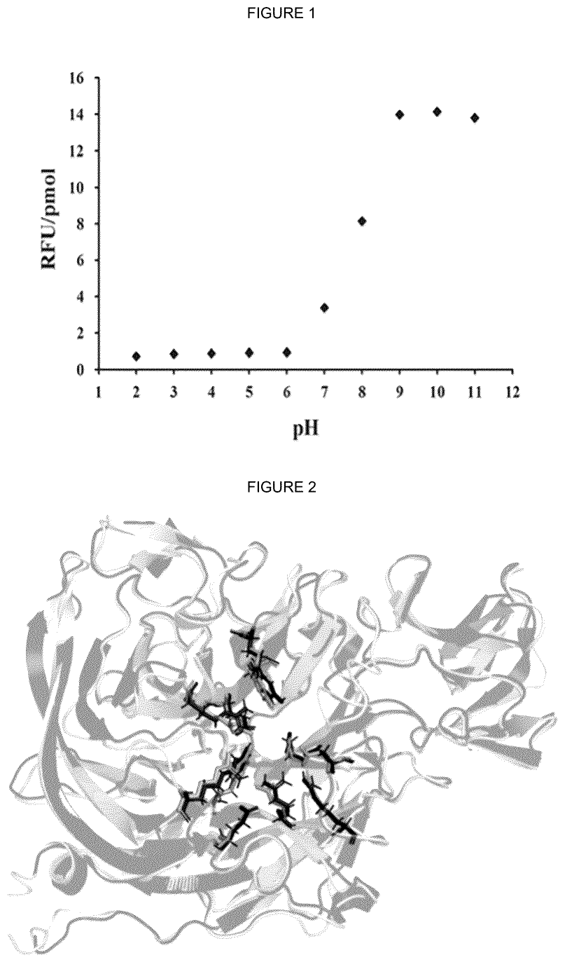

FIG. 1: Dependence of fluorescence response (in relative fluorescence units/pmol) of 4-methylumbelliferone in the pH range 2 to 11. Copied from: Profeta GS, Pereira JAS, Costa S G, et al. Standardization of a Continuous Assay for Glycosidases and Its Use for Screening Insect Gut Samples at Individual and Populational Levels. Frontiers in Physiology. 2017; 8:10.3389/fphys.2017.00308.

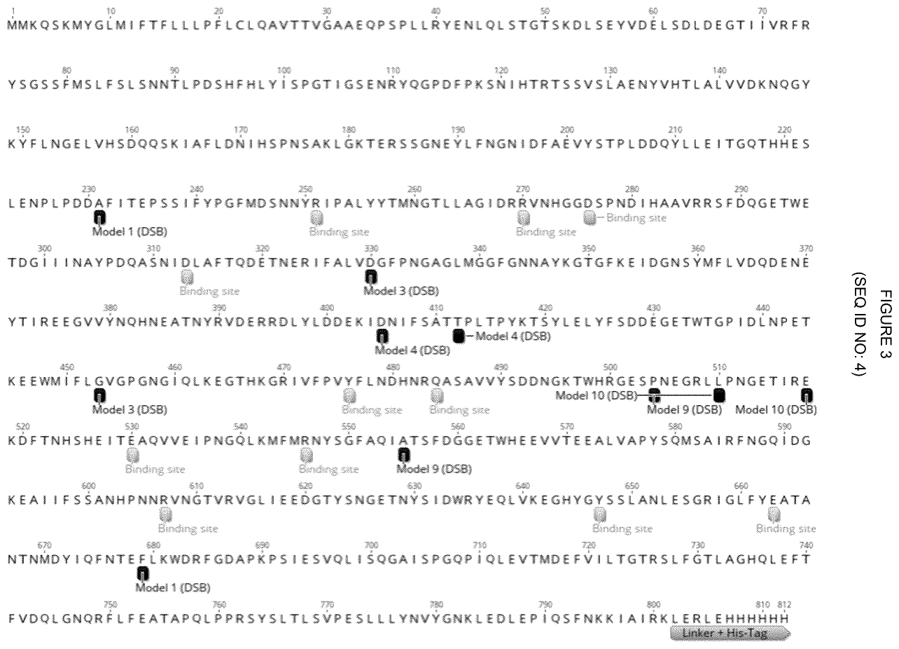

FIG. 2: Binding sites of AkNeu overlapping NanI. The cartoon in light gray represents the catalytic module of AkNeu and its binding pocket, whereas the cartoon and the binding pocket of NanI (PDB: 2BF6) are in dark gray. The cartoons of both enzymes are displayed with transparency to facilitate the visualization of the sticks of the binding pockets.

FIG. 3: Protein sequence of AkNeu (SEQ ID NO. 4) displaying the location of the proposed disulfide bridge mutants. From Geneious version 2023.1.2 created by Biomatters. Disulfide bridges (DSB) are highlighted in black. The pair of residues to be substituted with cysteines are identified for each model. The residues highlighted in light gray represent the predicted binding pocket in AkNeu based on alignment with the neuraminidase from Aspergillus fumigatus (PDB: 2XZI) and the sialidase from Clostridium perfringens (PDB: 2BF6).

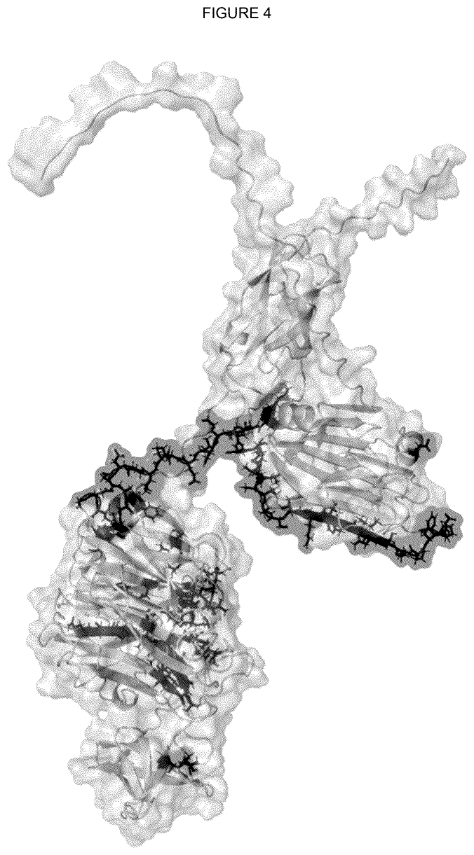

FIG. 4: AkNeu model generated with AlphaFold displaying the location of the proposed disulfide bridges and point mutation hotspots. The upper left module corresponds to the catalytic module identified in the core region of neuraminidases. From PyMOL version 2.5.2 created by Schrödinger. All proposed mutations (disulfide bridges and point mutations) are highlighted in black.

```
SEQ ID NO. 1: NanI WP_011590331.1 exo-alpha-sialidase NanI
[Clostridium perfringens]
    1 mnykgitlil taamvisggn yvlvkgstld sgknnsgyei kvnnsenlss lgeykdinle
  61 ssnasnityd lekyknldeg tivvrfnskd skiqsllgis nsktkngyfn fyvtnsrvgf
121 elrnqknegn tqngtenlvh mykdvalndg dntvalkiek nkgyklflng kmikevkdtn
181 tkflnnienl dsafigktnr ygqsneynfk gnigfmniyn eplgddylls ktgetkakee
241 vlvegavkte pvdlfhpgfl nssnyripal fktkegtlia sidarrqgga dapnndidta
301 vrrsedggkt wdegqiimdy pdkssvidtt liqddetgri fllvthfpsk ygfwnaglgs
361 gfknidgkey lclydssgke ftvrenvvyd kdgnkteytt nalgdlfrng tkidninsst
421 aplkakgtsy inlvysdddg ktwsepqnin fqvkkdwmkf lgiapgrgiq ikngehkgri
481 vvpvyytnek gkqssaviys ddsgknwtig espndnrkle ngkiinsktl sddapqltec
541 qvvempngql klfmrnlsgy lniatsfdgg atwdetvekd tnvlepycql svinysqkid
601 gkdavifsnp narsrsngtv riglinqvgt yengepkyef dwkynklvkp gyyaysclte
661 lsngniglly egtpseemsy iemnlkyles gank
SEQ ID NO. 2: NanA WP_014632427.1 LPXTG-anchored neuraminidase NanA
[Streptococcus pneumoniae]
    1 mnrsvqerkc rysirklsvg avsmivgavv fgtspvlaqe gaseqplane tqlsgesstl
  61 tdteksqpss etelsgnkqe qerkdkqeek iprdyyardl envetvieke dvetnasngq
121 rvdlsseldk lkklenatvh mefkpdakap afynlfsvss atkkdeyftm avynntatle
181 grgsdgkqfy nnyndaplkv kpgqwnsvtf tvekptaelp kgrvrlyvng vlsrtslrsg
241 nfikdmpdvt hvqigatkra nntvwgsnlq irnltvynra ltpeevqkrs qlfkrsdlek
301 klpegaalte ktdifesgrn gkpnkdgiks yripallktd kgtliagade rrlhssdwgd
361 igmvirrsed ngktwgdrvt itnlrdnpka sdpsigspvn idmvlvqdpe tkrifsiydm
421 fpegkgifgm ssqkeeaykk idgktyqily regekgayti rengtvytpd gkatdyrvvv
481 dpvkpaysdk gdlykgnqll gniyfttnkt spfriakdsy lwmsysdddg ktwsapqdit
541 pmvkadwmkf lgvgpgtgiv lrngphkgri lipvyttnnv shlngsqssr iiysddhgkt
601 whageavndn rqvdgqkihs stmnnrraqn testvvqlnn gdvklfmrgl tgdlqvatsk
661 dggvtwekdi krypqvkdvy vqmsaihtmh egkeyiilsn aggpkrengm vhlarveeng
721 eltwlkhnpi qkgefaynsl qelgngeygi lyehtekgqn aytlsfrkfn wdflskdlis
781 pteakvkrtr emgkgvigle fdsevlvnka ptlqlangkt arfmtqydtk tllftvdsed
841 mgqkvtglae gaiesmhnlp vsvagtklsn gmngseaavh evpeytgplg tsgeepaptv
901 ekpeytgplg tsgeepaptv ekpeytgplg tageeaaptv ekpeftggvn gtepavheia
961 eykgsdslvt lttkedytyk aplaqqalpe tgnkesdlla slgltafflg lftlgkkreq
SEQ ID NO. 3: AfNeu EAL89414.2 extracellular sialidase/neuraminidase,
putative [Aspergillus fumigatus Af293]
    1 mqsmrfmila llvqflpawa indpaksaap yhdefplfrs anmaspdkls tgigfhsfri
  61 pavvrtttgr ilafaegrrh tnqdfgdinl vykrtkttan ngaspsdwep lrevvgsgag
121 twgnptpvvd ddntiylfls wngatysqng kdvlpdgtvt kkidstwegr rhlyltesrd
181 dgntwskpvd ltkeltpdgw awdavgpgng irlttgelvi pamgrniigr gapgnrtwsv
241 qrlsgagaeg tivqtpdgkl yrndrpsqkg yrmvargtle gfgafapdag lpdpacqgsv
301 lrynsdapar tiflnsasgt srramrvris ydadakkfny grkledakvs gagheggyss
361 mtktgdykig alvesdffnd gtgknsyrai iwrrfnlswi lngpnn
SEQ ID NO. 4: AkNeu WP_084372094.1 sialidase family protein
[Halalkalibacter krulwichiae]
    1 mmkqskmygl miftflllpf lclqavttvg aaeqpspllr yenlqlstgt skdlseyvde
  61 lsdldegtii vrfrysgssf mslfslsnnt lpdshfhlyi spgtigsenr yqgpdfpksn
121 ihtrtssvsl aenyvhtlal vvdknqgyky flngelvhsd qqskiafldn ihspnsaklg
181 kterssgney lfngnidfae vystplddqy lleitgqthh eslenplpdd afitepssif
241 ypgfmdsnny ripalyytmn gtllagidrr vnhggdspnd ihaavrrsfd qgetwetdgi
301 iinaypdqas nidlaftqde tnerifalvd gfpngaglmg gfgnnaykgt gfkeidgnsy
361 mflvdqdene ytireegvvy nqhneatnyr vderrdlyld dekidnifsa ttpltpykts
421 ylelyfsdde getwtgpidl npetkeewmi flgvgpgngi qlkegthkgr ivfpvyflnd
481 hnrqasavvy sddngktwhr gespnegrll pngetirekd ftnhsheite aqvveipngq
541 lkmfmrnysg faqiatsfdg getwheevvt eealvapysq msairfngqi dgkeaiifss
601 anhpnnrvng tvrvglieed gtysngetny sidwryeqlv keghygyssl anlesgrigl
661 fyeatantnm dyiqfntefl kwdrfgdapk psiesvqlis qgaispgqpi qlevtmdefv
721 iltgtrslfg tlaghqleft fvdqlgnqrf lfeatapqlp prsysltlsv peslllynvy
781 gnkledlepi qsfnkkiair k
[]SEQ ID NO. 5: AhNeu GEN46640.1 hypothetical protein AHA02nite_24160
[Alkalibacillus haloalkaliphilus]
    1 mmlkgllptl fltalfllft spgafaeeie eekepilhve neeisggyft dinhkidelk
  61 eldqgtiivr frhegssfqs lfslsnnnfp nghfhlyvsp saigsenryq apgepqtnth
121 isqsltlend yvhtlamvvd qeegykyyln gelvledtts svqflnniye pnsaqlgrte
181 raaggnqytf hgdidfatvy geplqedylv eltgetares lenplpddam vtdpynvfyp
241 gfmdsnnyri palfytekgt liagidrrve sgadspndih sairrsydqg etwedegiii
301 naypddasni dlaftqdnsn erifalvdgf phgaglmggf gnnaypgtgy ttvngedymf
361 lldedeneyt ireegivydg egnptsykvd enrdlyldge kidnifsett plkpyktsyl
421 elyhsddegd nwtgpidlnp etkeewmifl gvgpgngiql segehegrii fpvyfinenl
481 rqasavvysd dggetwqrge spnqgrvvdg nvlderyfds pnheiteaqv vempdgqlkm
541 fmrnysgyaq vatsydgget wdsevitetd lvapysqmsa irydgqidgk davifssand
601 htsringtvr vglieengpl engftdysfd wkyeqlvkeg hygyssltnl pdgelglfye
661 gtpntvmdfi kfntdflkwe rhieeptpel inythlkqep nvyrkgdsvk velefddfvv
721 lmgdqtlkge idghaikfnl vesnnssftf eatlpdlkpr aydinlefss dltiynkygk
781 lldtvylhfd ssekikvtag nhne
SEQ ID NO. 6: AsNeu WP_059104496.1 sialidase family protein [Shouchella
shacheensis]
    1 mttklfkflf vmtmallivl qvgedsptfa ktkepvlrve nqaivdnqfv nledevgqlk
  61 eldegtiivr frhtgssfms lislsnknfp dshfhfyisp sgvgsenrye apgepkenih
121 vqsdsltlqe gevytvamvm dkgegykyfl dgelikedtq sprkflsnvy epnsaylgqt
181 drsqgnsypf ngdidfaevh seplsdgvle ditgetaksd pvtnpmpdda fvsepesify
241 pgfmdspnfr ipalyyteng tllagidrrv ggggdspndi haavrrsldq gdtweddgil
301 inaypddasn idlsfvqdqs servfalvdg wpagaglmgg fgtntykgtg fetidgkdym
361 fltdeddnry tireegkvyd eggnstdysv dternlfqng eeidnifsqt splkpfktsy
421 lelyysddeg etwtgpidln dqtkeeymmf lgvgpgngiq lnegsnkgrl vfpvyfindn
481 lrqasaviys ddngetwhrg espnegrdvg dgeiineedf tnqaheitea qvvempdgql
541 kmfmrnysgy aqiatsfdgg etwdteivte tdlvapysqm sairydgqvd gqeavvfssa
601 nhptsridgt vkiglieedg ehkngytkys fdwkyeqlvk qghygysslt nlpdgeigll
661 yegtantemd fmkfntdfvk wdrveerpqp elesmnviss pppvhrsgek iqieatfddy
721 vmligdkrlv gtiageevhf dlvdrtgqdh fvfeaevpel pprthqleae fdsdltiynl
781 ygkrfseetk ladtirvrat grggntfsms n
```

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

### EXAMPLES

### Example 1

In the context of the present invention, the inventors established neuraminidase as possible enzyme to be used for Immunohistochemical (IHC) staining. Potentially, it could be used as orthologous system in parallel to well established enzymes like Alkaline Phosphatase or Horseradish Peroxidase allowing for multiplexed IHC approaches. Neuraminidases, also called sialidases, belong to the family of glycoside hydrolases and catalyses the hydrolysis of N-acetylneuraminic acid (or sialic acid) from an adjacent glycoprotein, glycolipids and polysaccharides. MU-NANA (2′-(4-methylumbelliferyl)-α-D-N-acetylneuraminic acid) is a well-established fluorescent substrate for glycoside hydrolase assays, and it has been selected as screening substrate in this study. The hydrolysis of MU-NANA produces N-acetylneuraminic acid and 4-methylumbelliferone, a blue, fluorescent and soluble molecule that can be monitored fluorometrically. The fluorescence response of the reporter 4-methylumbelliferone is pH-dependent and allows meaningful signal detection only at pH values higher than 8 (see FIG. 1). Consequently, alkaline pH (ideally pH 9.0-9.5) is required for optimal compatibility with enzymes used in a multiplex IHC assay.

To establish a neuraminidase for IHC applications, the inventors first performed a research study by reviewing the literature to identify alternative enzymes active at alkaline pH value. In a next step, data mining using as target the previously selected enzymes was conducted to identify new scaffolds suitable for this reaction condition. Later, the inventors further optimized the activity at pH 8-10, the substrate affinity (KM), the substrate turnover (kcat) and the substrate specificity (kcat/KM) by introducing structure-guided mutations.

Surprisingly, two disulfide-stabilized mutants gave rise to a neuraminidase with desired features while other disulfide-bridge mutants as well as several point mutations failed to provide required activity. There were 79 positions out of 801 residues tested, and 5 alternative disulfide bridges evaluated, covering most of the enzyme sequence.

### Example 2: Candidate Selection Based on Scaffold Characteristics

Based on the results of the research study, the inventors used the well-known NanI from Clostridium Perfringens as the starting point of this study. This enzyme was tested using MU-NANA among other neuraminic acid derivatives as a proof of concept for the intended application. Although this enzyme displayed the desired activity and substrate acceptance with all the substrates evaluated, it lacked a good performance at high pH values (almost no activity at alkaline conditions and low stability).

Thus, this structure was selected as the template for data mining to find more candidates with improved performance for the intended operating conditions. Among the specific filters selected to identify new scaffolds, B-factor for some motifs of the enzyme, sources and length of the enzyme, and its suitability to be conjugated were prioritized.

Among the sequences identified after in silico analysis, six candidates were cloned, expressed, biochemically characterized and tested using the target operating conditions. In addition to the good to excellent performance under alkaline pH, it was desired a good organic solvent tolerance (e.g., DMSO) and high turnover of the substrate.

The coding gene for each neuraminidase was cloned in a derivative vector of pQE80. A 5-mL culture of E. coli BL21 hosting this plasmid with an initial OD600 of 0.1 was cultivated in Magic Media (ThermoFisher) and supplemented with an antibiotic for 3 h at 37° C. and 250 rpm. The protein expression was induced using IPTG 0.1 mM. Since the final OD600 for the candidates oscillated between 0.4 and 0.6, the cell concentration was normalized before the activity test.

It is important to remark that the expression profile of all of candidates was similar. The cells were harvested (3000 rpm, 10 min, 4° C.) and resuspended in the desired buffer (for instance, glycine 200 mM, pH 9.5 for the target condition), supplemented with B-PER at a final concentration of 4%. The cell disruption was carried out at room temperature for 30 minutes and 600 rpm. The samples were centrifuged (4700 rpm, 10 min, 4° C.), and the supernatant was implemented for further experimentation.

The kinetic parameters were determined for 60 minutes according to a methodology similar to Marathe et al., 2013. The initial screening determined the optimum temperature, although 33° C. was set as the target temperature for further evaluations since it constitutes the intended operating conditions. The reaction mixture combined 25 μL of 1 mM MU-NANA dissolved in water, 24 μL of supernatant (for instance, B-PER 4%, glycine 200 mM, pH 9.5 for the target condition) and 1 μL of DMSO. The excitation and emission wavelengths were 355 nm and 460 nm, respectively, and the gain was set at 600.

The kinetics parameters were calculated based on the maximum slope (initial velocity), maximum fluorescence intensity (maximum velocity), time to the maximum slope and time to the plateau. The biochemical characterization of the candidates implemented MU-NANA as substrate considering the temperature profile, organic solvent tolerance, salt requirements, buffer condition and pH profile. It is important to highlight that the operating condition in the range of the intended application was also evaluated (i.e., 33±3° C., pH value 9.0-9.5, 1-5% DMSO). The comparison of all the above-mentioned parameters among those enzymes revealed the most promising candidates. The following table displays the main biochemical features identified in our screening for all those candidates (compilation of data generated in-house and public information).

TABLE 1
Biochemical characterization of neuraminidases and identification of key features of their performance.

| | | NanI | | NanA | | AfNeu | | AkNeu | | AhNeu | | AsNeu | |
|---|---|---|---|---|---|---|---|---|---|---|---|---|---|
| Incubation | Temperature | 28° | C. | 28° | C. | 28° | C. | 28° | C. | 28° | C. | 28° | C. |
| Disruption | Time | 30 | min | 30 | min | 30 | min | 30 | min | 30 | min | 30 | min |
| | Temperature | Room | | Room | | Room | | Room | | Room | | Room | |
| | | temperature | | temperature | | temperature | | temperature | | temperature | | temperature | |
| | Buffer | Tris 100 mM, | | Tris 100 mM, | | pH 4.0-10.0, | | Tris 100 mM, | | Tris 100 mM, | | Tris 100 mM, | |
| | | pH 7.2, | | pH 8.4, | | B-PER 4% | | pH 8.5, | | pH 8.5, | | pH 7.0, | |
| | | B-PER 4% | | B-PER 4% | | | | B-PER 4% | | B-PER 4% | | B-PER 4% | |
| Optimal | Temperature | 45° | C. | 45° | C. | nd | | 43° | C. | 43° | C. | 40° | C. |
| reaction | Buffer | Tris, | | Tris, | | nd | | Tris, | | Tris, | | Tris, | |
| | | pH 7.0-7.3 | | pH 8.0-8.4 | | | | pH 8.0-8.5 | | pH 7.5-8.5 | | pH 7.0-7.4 | |
| | dFI/s (initial | 174 | | 34 | | nd | | 174 | | 40 | | nd | |
| | velocity) | | | | | | | | | | | | |
| | Time to plateau | 10 | min | 15 | min | nd | | 10 | min | 15 | min | nd | |
| Screening | Substrate | MU-NANA | | MU-NANA | | MU-NANA | | MU-NANA | | MU-NANA | | MU-NANA | |
| | | 50 μM | | 50 μM | | 50 μM | | 50 μM | | 50 μM | | 50 μM | |
| | Buffer | Tris 100 mM, | | Tris 100 mM, | | nd | | Glycine 100 mM, | | Tris 100 mM, | | Tris 100 mM, | |
| | | pH 8.2 | | pH 8.2 | | | | pH 9.5 | | pH 9.0 | | pH 8.0 | |
| | DMSO | nd | | nd | | nd | | ≤4% | | nd | | nd | |
| Readout | Sensitivity | 106 | | 92 | | nd | | 81 (144 without | | 81 | | nd | |
| | (FIₘₐₓ/C—) | | | | | | | DMSO) | | | | | |
| | Temperature | 45° | C. | 45° | C. | 37° | C. | 33° | C. | 43° | C. | 40° | C. |

| Features | Molecular | 78.9 | 114.3 | 45.9 | 91.6 | 92.2 | 92.3 |
|---|---|---|---|---|---|---|---|
| | weight (kDa)* | | | | | | |
| | Isoelectric point* | 5.45 | 6.06 | 9.65 | 4.46 | 4.05 | 4.08 |
| | Length (aa)* | 705 | 1031 | 417 | 812 | 815 | 822 |
| | Microorganism° | Clostridium | Streptococcus | Aspergillus | Halalkalibacter | Alkalibacillus | Shouchella |
| | | perfringens | pneumoniae | fumigatus | krulwichiae | haloalkaliphilus | shacheensis |
| | | ATCC 13124 | | Af293 | | | |
| | GenBank° | WP_011590331 | WP_014632427 | EAL89414.2 | WP_084372094.1 | GEN46640.1 | WP_059104496 |

*The parameters were calculated with Geneious version 2023.1.2 created by Biomatters.
°The NCBI reference sequences (GenBank) and the host of the wild-type enzymes (microorganism) are reported in the NCBI.

Since the alkaline operating conditions corresponded to the target, the first characterization was the pH profile of each enzyme. Different buffers with pH values from 4.0 to 10.0 were evaluated. After the experiment, the candidate AfNeu (see Table 1) was excluded right away as unsuitable for the inventors desired application because of the low acceptance of the substrates. Similarly, AsNeu was discarded after determining the optimum pH (it displays similar operating conditions to NanI, pH 7.0 to 7.4).

The rest of the experiments were performed only with NanA, AkNeu and AhNeu. It became evident that after comparing the turnover (initial velocity), time to maximum velocity (time to plateau) and pH optimum, that AkNeu constituted the best scaffold for the inventor's final application (i.e., 174 dFI/s, 10 min, pH 8.0-8.5, respectively). As a final biochemical characterization, different DMSO concentrations from 1% to 5% in the final solutions were tested using the target and the optimum operating conditions. It was determined a good-to-high DMSO tolerance for AkNeu without affecting drastically the enzyme performance under both conditions.

Although the organic solvent tolerance is important for substrate solubility in the final platform, it is not the critical criterion to discard or select a given backbone since it is possible to achieve via protein engineering. In contrast, selecting the right backbone with an already close optimum pH value to the target condition was considered as the crucial criterion.

Table 1 summarizes the results of the experimental screening, and although AkNeu was the most promising candidate, this scaffold needed engineering to optimize the stability and the pH optimum range for the target operating conditions.

### Example 3: Structure-Guided Mutations of AkNeu

The binding pocket was identified based on structural alignments of the AkNeu amino acid sequence (SEQ ID NO 4) with the X-ray structures of neuraminidase from Aspergillus fumigatus (AfNeu) (PDB: 2XZI) and the sialidase from Clostridium perfringens (NanI (PDB: 2BF6) focusing on the alignment of the core region (using the available tool from 3DM, Bio-Prodict B.V.).

Based on this structural alignment, the inventors could infer that the following residues are likely to form the AkNeu substrate-binding site: R251, R270, D276, D313, Y476, Q484, E530, R546, R607, Y647, and E663. When aligned to NanI (PDB: 2BF6) the catalytic domain of AkNeu showed high structural similarity to NanI, even with a similar spatial arrangement of the catalytic residues (FIG. 2).

With the structural alignment at hand, the inventors set out to identify sites that would be suitable to engineer a disulfide bridge that might further stabilize the AkNeu protein. The prediction of the disulfide bridges was based on the B-factor of residues and protein regions, the potentiality to form disulfide bridges after the interaction of the cysteines, and the probability of reducing the conformational entropy.

Thus, two crystals of the neuraminidase from Clostridium perfringens (PDBs 2BF6 and 5TSP) were analyzed simultaneously with relaxed models of AkNeu generated with AlphaFold, and those results were contrasted in order to identify consensuses among them. The candidates were ranked considering the higher probability of improving protein stability without modifying drastically the binding site or protein folding. Once again, the core region was implemented to understand, compare and visualize the overlapping regions for all the proposed mutations (FIG. 3). Five models, each containing one predicted disulfide bridge, were selected for further experimentation: A231C-F679C (Model 1), D330C-G453C (Model 3), D405C-T412C (Model 4), P504C-A555C (Model 9), and L510C-E518C (Model 10).

Furthermore, the inventors used the structural model to identify sites for point mutations that might have a positive effect on AkNeu performance. Point mutations considered the residues with higher B-factor and RMSD values in the sequence, as well as their correlated mutations. Thus, 70 hotspots were identified fulfilling these criteria (FIG. 4). Additionally, 8 extra residues (so-called, bridging positions) were selected because they were single connectors between hotspot regions. Site-saturation mutagenesis was performed in those residues using NNK mutagenesis. The following positions were targeted (bridging positions are underlined): D63, G113, P114, D115, F116, P117, K118, 5119, N120, 1121, H122, T123, R124, T125, 5126, S127, V128, S129, L130, A131, E132, N133, Y134, G216, Q217, T218, H219, H220, E221, S222, L223, E224, N225, P226, L227, P228, D229, D230, A231, F232, G266, D268, A309, S310, N311, 1312, D313, L314, A315, F316, T317, Q318, D330, G331, F332, F451, L452, G453, K542, A552, L574, P577, M581, F586, 1590, D591, G592, A595, T611, G615, L616, 1617, D620, S631, S649, T668, N669, and M670. Both, disulfide bridges and point mutations that could compromise the binding pocket were discarded in this screening (data not shown).

### Example 4: Activity Tests at Alkaline pH

The above-mentioned positions (point mutations and disulfide bridges) were tested at the target operating conditions (i.e., 33° C. and pH 9.5). The recombinant mutants for the point mutations were generated using degenerated primers containing NNK (forward) and MNN (reverse) codons according to Zheng et al., 2004, and transformed in E. coli BL21, similar to the wild-type enzyme. One microtiter plate was picked for each point mutation selected. In the case of the double mutants to generate the disulfide bridges, the plasmids were purchased with the respective mutations codifying for cysteines.

The hit detection among those mutants implemented MU-NANA as the substrate to identify improvements in the mutant performance (activity and stability). The evaluation was similar to the biochemical characterization described before. The incubation time for enzyme expression and the reaction mixture were optimized beforehand to guarantee the exponential phase of the reactions (i.e., initial velocity) during the screening. The set screening condition facilitates the visualization of small differences of performances among the mutants and therefore identifies accurately improvements or detrimental effects.

Each mutant was cultivated in 250 μL of Magic Media supplemented with the antibiotic and IPTG 0.1 mM in a 96-well format plate. The microtiter was incubated for 20 h at 28° C. and 250 rpm. The cells were harvested (3000 rpm, 10 min, 4° C.) and resuspended in the desired buffer for the target condition (glycine 200 mM, pH 9.5), supplemented with 4% B-PER. The cell disruption was carried out at room temperature for 30 minutes and 600 rpm, the lysed samples were centrifuged (4700 rpm, 10 min, 4° C.), and the supernatant was used during screening.

The enzymatic activity was measured for 60 minutes at 33° C. The reaction mixture combined 25 μL of 1 mM MU-NANA dissolved in water, 24 μL of supernatant (B-PER 4%, glycine 200 mM, pH 9.5) and 1 μL of DMSO. The excitation and emission wavelengths were 355 nm and 460 nm, respectively. Improvements in the performance considered kinetic parameters such as the maximum slope (initial velocity), maximum fluorescence intensity (maximum velocity), time to the maximum slope and time to the plateau.

Many of the evaluated positions did not affect considerably the enzyme performance, and only a few played a detrimental effect under the evaluation conditions. On the other hand, a few mutants displayed better stability at pH 9.5 in comparison to wt-AkNeu. Those results were confirmed using replicates (four to eight replicates) and evaluating them at different pH values between 8.0 and 9.5. Among those improved mutants, two disulfide bridge models and a few point mutations were identified displaying better performance at pH values higher than 9.0 in comparison to wt-AkNeu.

### Example 5: Enzyme Kinetic Analysis (Kcat, KM, Kcat/KM)

Those clones displaying stability improvements in comparison to the wild-type enzyme were biochemically characterized to determine the kinetic parameters and estimate the real improvement. This characterization confirmed that those improvements were not due to higher expression levels, whereas the substrate acceptance and turnover were either not affected or had detrimental consequences. The ideal hit to be used in IHC should display higher stability and the kinetic parameters must remain as the wild-type enzyme or improve them.

The candidates and wt-AkNeu were cultivated in 1000 mL Magic Media supplemented with the antibiotic for 3 h at 37° C. and 250 rpm. Later, the protein expression was induced using 0.1 mM IPTG and the cells were incubated overnight at the same operating conditions. The cells were harvested (5000 rpm, 30 min, 4° C.) and resuspended in KH₂PO₄ 20 mM, KCl 150 mM, pH 7.0 (buffer:biomass, 1:3) for 30 minutes. The cells were disrupted in a French Press at 1.5 Kbar and later centrifuged for 1 h at 11000 rpm. The resulting cellular extract was pre-treated with 1-3% Polymin-G20 and centrifuged again (11000 rpm, 60 min, 4° C.).

The clear supernatant was loaded on a Ni-NTA column (cOmplete, Roche). The His-tagged protein was washed with KH₂PO₄ 20 mM, KCl 150 mM and imidazole 2 mM, pH 7.0, and eluted with a gradient from 2 mM to 250 mM imidazole using 10 column volumes. The fractions were analyzed in SDS-PAGE and with the activity test using MU-NANA as the substrate. The fractions containing the enzyme in higher proportion were pooled, concentrated and dialyzed (10-kDa molecular weight cutoff) using MES 20 mM, pH 6.0. The sample was diluted until the conductivity was under 3 mS/cm before it was loaded on a Q-Sepharose XL (Cytiva) column. The protein was eluted with a 20-column-volume gradient from 0 mM to 1000 mM NaCl. Again, the protein content was analyzed in SDS-PAGE and with MU-NANA. The fractions containing the enzyme with higher quality were pooled, concentrated, and later analyzed in UPLC (Vanquish Horizon, Thermo Fisher) using a column TSKgel UP-SW3000 (Tosoh Biosciences) and PBS, pH 7.0 (Roche) as mobile phase. The obtained sample for each sample was adjusted to a final concentration of 1 mg/mL and these samples were implemented for further experimentation (e.g., enzyme conjugation and kinetic parameters determination).

The activities of the purified wt-AkNeu and the other candidates were tested using a few sialic acid derivatives and MU-NANA. No difference in the substrate acceptance was detected. Table 2 displays the kinetic parameters of wt-AkNeu using MU-NANA at 33° C. (target temperature) and 42° C. (optimum temperature).

TABLE 2
Biochemical profile of wt-AkNeu.

| Temperature | Vmax | | | kcat/KM [s⁻¹ |
|---|---|---|---|---|
| (° C.) | [μM/s] | kcat [s⁻¹] | KM [mM] | mM⁻¹] |
| 33 | 0.06383 | 5.978 | 0.1264 | 47.30 |
| 42 | 0.06907 | 6.469 | 0.1185 | 54.59 |

Table 3 displays a summary of the top 5 candidates tested at 33° C. It is important to highlight that the optimal temperature for each mutant remains intact after the introduction of the mutations (data not shown).

TABLE 3
Biochemical characterization of the top 5 mutants identified during the screening.
The parameters improved in comparison to wt-AkNeu are highlighted in bold.

| | Relative activity in comparison | | | Kinetic parameters respect to | | |
|---|---|---|---|---|---|---|
| | to pH 8.0* and 33° C. | | | wt-AkNeu (pH 9.5, 33° C.) | | |
| | pH 9.0 | pH 9.2 | pH 9.5 | kcat | KM | kcat/KM |
| wt-AkNeu | 72% | 55% | 53% | 100% | 100% | 100% |
| AkNeu-D405C- | 85% | nd | 65% | 110% | 119% | 93% |
| T412C | | | | | | |
| AkNeu-A231C- | 74% | nd | 56% | 103% | 99% | 105% |
| F679C | | | | | | |
| AkNeu-P228A | 88% | 75% | 71% | 61% | 100% | 62% |
| AkNeu-E221R | 80% | 51% | 47% | 46% | 71% | 65% |
| AkNeu-D115N | 82% | 61% | 52% | 32% | 119% | 27% |

*The activity of a mutant at a given pH value was contrasted with the activity of the same mutant at pH 8.0 and 33° C., a condition in which wt-AkNeu shows 100% activity for the intended application.
° The kinetic parameters of a mutant were compared with the activity of wt-AkNeu at pH 9.5 and 33° C. (intended application).

At pH 9.0 the mutants AkNeu-D405C-T412C, AkNeu-A231C-F679C, AkNeu-P228A, AkNeu-E221R, AkNeu-D115N showed a higher activity relative to wt-AkNeu.

At the target pH 9.5 the double mutants D405C-T412C and A231C-F679C as well as the point mutant P228A show increased enzymatic activity compared to wt AkNeu.

### REFERENCES

1. Ortiz Hidalgo C (2022), Del Valle L (ed.), “Immunohistochemistry in Historical Perspective: Knowing the Past to Understand the Present”, Immunohistochemistry and Immunocytochemistry, Methods in Molecular Biology, vol. 2422, New York, NY: Springer US, pp. 17-31, doi:10.1007/978-1-0716-1948-3_2.
2. Duraiyan J, Govindarajan R, Kaliyappan K, Palanisamy M (2012). “Applications of immunohistochemistry”. Journal of Pharmacy and Bioallied Sciences. 4 (6): S307-9. doi:10.4103/0975-7406.100281.
3. Magaki S, Hojat S A, Wei B, So A, Yong W H (2019), Yong W H (ed.), “An Introduction to the Performance of Immunohistochemistry”, Biobanking, vol. 1897, New York, NY: Springer New York, pp. 289-298, doi:10.1007/978-1-4939-8935-5_25.
4. Ramos-Vara JA (2005). “Technical Aspects of Immunohistochemistry”. Veterinary Pathology. 42 (4): 405-426. doi:10.1354/vp.42-4-405.
5. Magaki S, Hojat S A, Wei B, So A, Yong W H (2019), Yong W H (ed.), “An Introduction to the Performance of Immunohistochemistry”, Biobanking, vol. 1897, New York, NY: Springer New York, pp. 289-298, doi:10.1007/978-1-4939-8935-5_25.
6. Krenacs T, Krenacs L, Raffeld M (2010), Oliver C, Jamur M C (eds.), “Multiple Antigen Immunostaining Procedures”, Immunocytochemical Methods and Protocols, vol. 588, Totowa, NJ: Humana Press, pp. 281-300, doi:10.1007/978-1-59745-324-0_28.
7. Miyagi T, Yamaguchi K. (2012), “Mammalian sialidases: physiological and pathological roles in cellular functions.” Glycobiology 22:880-896.
8. Amith S R, Jayanth P, Franchuk S, Finlay T, Seyrantepe V, Beyaert R, Pshezhetsky A V, Szewczuk M R. (2010), “Neul desialylation of sialyl alpha-2,3-linked beta-galactosyl residues of TOLL-like receptor 4 is essential for receptor activation and cellular signaling.” Cell Signal 22:314-324.
9. Profeta G S, Pereira J A S, Costa S G, et al. (2017), “Standardization of a Continuous Assay for Glycosidases and Its Use for Screening Insect Gut Samples at Individual and Populational Levels.” Frontiers in Physiology 8:10.3389/fphys.2017.00308.
10. Marathe, B. M., et al. (2013). “Determination of neuraminidase kinetic constants using whole influenza virus preparations and correction for spectroscopic interference by a fluorogenic substrate.” PLOS ONE 8(8): e71401.
11. Zheng L, Baumann U, Reymond JL. (2004), “An efficient one-step site-directed and site-saturation mutagenesis protocol.” Nucleic Acids Res. 32(14):e115. doi: 10.1093/nar/gnh110.

1. A recombinant mutated neuraminidase enzyme or a functional fragment thereof, comprising an amino acid sequence having at least 85% sequence identity to SEQ ID NO. 4, further comprising at least one mutation between residues 115 and 680 of SEQ ID NO: 4, wherein said recombinant mutated neuraminidase enzyme or functional fragment thereof exhibits an increased enzymatic activity at alkaline pH when compared to the non-mutated neuraminidase enzyme.

2. The enzyme or the functional fragment thereof according to claim 1, further comprising at least one additional mutation between residues 400 and 420 of SEQ ID NO: 4, wherein said recombinant mutated neuraminidase enzyme or functional fragment thereof further exhibits an increased kinetic activity at alkaline pH when compared to the non-mutated neuraminidase enzyme.

3. The enzyme or the functional fragment thereof according to claim 1, wherein the neuraminidase enzyme is derived from the sialidase family protein of the bacterium Halalkalibacter krulwichiae or a bacterial homolog thereof having neuraminidase activity.

4. The enzyme or the functional fragment thereof according to claim 1, wherein the position of the at least one mutation is selected from the group consisting of positions 115, 221, 228, 231, 405, 412, and 679 according to SEQ ID NO: 4.

5. The enzyme or the functional fragment thereof according to claim 1, wherein the mutation is selected from a substitution, deletion, or amino acid modification.

6. The enzyme or the functional fragment thereof according to claim 1, wherein the at least one mutation is selected from the group consisting of D115N, E221R, P228A, A231C, D405C, T412C, and F679C.

7. The enzyme or the functional fragment thereof according to claim 1, wherein the amino acid sequence of the recombinant mutated neuraminidase enzyme or the functional fragment thereof comprises two mutations selected from A231C and F679C, and D405C and T412C.

8. The enzyme or the functional fragment thereof according to claim 1, wherein the recombinant mutated neuraminidase enzyme, or the functional fragment thereof, exhibits an increased enzymatic activity at a pH range of between 8.0 to 10 compared to the non-mutated neuraminidase enzyme.

9. A nucleic acid encoding the enzyme or the functional fragment thereof according to claim 1, or a vector comprising the nucleic acid.

10. The vector according to claim 9, which is an expression vector.

11. A recombinant cell, comprising the enzyme or the functional fragment thereof according to claim 1.

12. A method for producing the enzyme or the functional fragment thereof according to claim 1, comprising: i) Culturing and harvesting a recombinant cell expressing the enzyme or the functional fragment thereof, and ii) suitably isolating the enzyme or the functional fragment thereof as expressed.

13. A method for immunohistochemical (IHC) staining of a biological target comprising i) suitably coupling or complexing the recombinant mutated neuraminidase enzyme or a functional fragment thereof according to claim 1 to a suitable binder to produce a tagged binder ii) contacting the tagged binder with a sample suspected to contain the biological target together with a suitable chromogenic substrate for the enzyme at an alkaline pH, iii) immunohistochemical (IHC) staining of the biological target, and optionally iv) detecting the immunohistochemical (IHC) staining.

14. A kit for performing an immunohistochemical (IHC) staining, comprising the enzyme or a functional fragment thereof according to claim 1, optionally coupled to an antibody or fragment thereof, a suitable substrate for the enzyme or fragment thereof, and additional materials for immunohistochemical (IHC) staining.

15. The enzyme or the functional fragment thereof according to claim 1, wherein the amino acid sequence has at least 90% sequence identity to SEQ ID NO. 4.

16. The enzyme or the functional fragment thereof according to claim 3, wherein the neuraminidase enzyme is derived from the sialidase family protein of the bacterium Halalkalibacter krulwichiae according to SEQ ID NO: 4 or a bacterial homolog thereof having neuraminidase activity

17. The enzyme or the functional fragment thereof according to claim 8, wherein the recombinant mutated neuraminidase enzyme, or the functional fragment thereof, exhibits an increased enzymatic activity at a pH range of between 8.5 to 9.5 or a pH range of between 9.0 to 9.5 compared to the non-mutated neuraminidase enzyme.

18. The vector according to claim 10, which is an expression vector comprising a promoter sequence operably linked to the nucleic acid.

19. The method of claim 13, wherein the biological target is a cell or tissue section, wherein the suitable binder is an antibody or antigen binding fragment thereof, and wherein the suitable chromogenic substrate for the enzyme is selected from the group consisting of 5-Carboxytetramethylrhodamin (5-TAMRA), Cyanine5 (Cy5), Dibenzocyclooctyne-Cy5 (DBCO-Cy5), and 4-(4-Dimethyl-aminophenylazo)benzolsulfonylchlorid (Dabsylchlorid).

20. The enzyme or the functional fragment thereof according to claim 2, comprising two mutations between residues 400 and 420 of SEQ ID NO: 4.


SEQUENCE LISTING (6 sequences)

SEQ ID NO: 1 - protein, 694 aa, Clostridium perfringens
MNYKGITLILTAAMVISGGNYVLVKGSTLDSGKNNSGYEIKVNNSENLSSLGEYKDINLESSNASNITYDLEKYKNLDEGTIVVRFNSKDSKIQSLLGISNSKTKNGYFNFYVTNSRVGFELRNQKNEGNTQNGTENLVHMYKDVALNDGDNTVALKIEKNKGYKLFLNGKMIKEVKDTNTKFLNNIENLDSAFIGKTNRYGQSNEYNFKGNIGFMNIYNEPLGDDYLLSKTGETKAKEEVLVEGAVKTEPVDLFHPGFLNSSNYRIPALFKTKEGTLIASIDARRQGGADAPNNDIDTAVRRSEDGGKTWDEGQIIMDYPDKSSVIDTTLIQDDETGRIFLLVTHFPSKYGFWNAGLGSGFKNIDGKEYLCLYDSSGKEFTVRENVVYDKDGNKTEYTTNALGDLFRNGTKIDNINSSTAPLKAKGTSYINLVYSDDDGKTWSEPQNINFQVKKDWMKFLGIAPGRGIQIKNGEHKGRIVVPVYYTNEKGKQSSAVIYSDDSGKNWTIGESPNDNRKLENGKIINSKTLSDDAPQLTECQVVEMPNGQLKLFMRNLSGYLNIATSFDGGATWDETVEKDTNVLEPYCQLSVINYSQKIDGKDAVIFSNPNARSRSNGTVRIGLINQVGTYENGEPKYEFDWKYNKLVKPGYYAYSCLTELSNGNIGLLYEGTPSEEMSYIEMNLKYLESGANK

SEQ ID NO: 2 - protein, 1020 aa, Streptococcus pneumoniae
MNRSVQERKCRYSIRKLSVGAVSMIVGAVVFGTSPVLAQEGASEQPLANETQLSGESSTLTDTEKSQPSSETELSGNKQEQERKDKQEEKIPRDYYARDLENVETVIEKEDVETNASNGQRVDLSSELDKLKKLENATVHMEFKPDAKAPAFYNLFSVSSATKKDEYFTMAVYNNTATLEGRGSDGKQFYNNYNDAPLKVKPGQWNSVTFTVEKPTAELPKGRVRLYVNGVLSRTSLRSGNFIKDMPDVTHVQIGATKRANNTVWGSNLQIRNLTVYNRALTPEEVQKRSQLFKRSDLEKKLPEGAALTEKTDIFESGRNGKPNKDGIKSYRIPALLKTDKGTLIAGADERRLHSSDWGDIGMVIRRSEDNGKTWGDRVTITNLRDNPKASDPSIGSPVNIDMVLVQDPETKRIFSIYDMFPEGKGIFGMSSQKEEAYKKIDGKTYQILYREGEKGAYTIRENGTVYTPDGKATDYRVVVDPVKPAYSDKGDLYKGNQLLGNIYFTTNKTSPFRIAKDSYLWMSYSDDDGKTWSAPQDITPMVKADWMKFLGVGPGTGIVLRNGPHKGRILIPVYTTNNVSHLNGSQSSRIIYSDDHGKTWHAGEAVNDNRQVDGQKIHSSTMNNRRAQNTESTVVQLNNGDVKLFMRGLTGDLQVATSKDGGVTWEKDIKRYPQVKDVYVQMSAIHTMHEGKEYIILSNAGGPKRENGMVHLARVEENGELTWLKHNPIQKGEFAYNSLQELGNGEYGILYEHTEKGQNAYTLSFRKFNWDFLSKDLISPTEAKVKRTREMGKGVIGLEFDSEVLVNKAPTLQLANGKTARFMTQYDTKTLLFTVDSEDMGQKVTGLAEGAIESMHNLPVSVAGTKLSNGMNGSEAAVHEVPEYTGPLGTSGEEPAPTVEKPEYTGPLGTSGEEPAPTVEKPEYTGPLGTAGEEAAPTVEKPEFTGGVNGTEPAVHEIAEYKGSDSLVTLTTKEDYTYKAPLAQQALPETGNKESDLLASLGLTAFFLGLFTLGKKREQ

SEQ ID NO: 3 - protein, 406 aa, Aspergillus fumigatus
MQSMRFMILALLVQFLPAWAINDPAKSAAPYHDEFPLFRSANMASPDKLSTGIGFHSFRIPAVVRTTTGRILAFAEGRRHTNQDFGDINLVYKRTKTTANNGASPSDWEPLREVVGSGAGTWGNPTPVVDDDNTIYLFLSWNGATYSQNGKDVLPDGTVTKKIDSTWEGRRHLYLTESRDDGNTWSKPVDLTKELTPDGWAWDAVGPGNGIRLTTGELVIPAMGRNIIGRGAPGNRTWSVQRLSGAGAEGTIVQTPDGKLYRNDRPSQKGYRMVARGTLEGFGAFAPDAGLPDPACQGSVLRYNSDAPARTIFLNSASGTSRRAMRVRISYDADAKKFNYGRKLEDAKVSGAGHEGGYSSMTKTGDYKIGALVESDFFNDGTGKNSYRAIIWRRFNLSWILNGPNN

SEQ ID NO: 4 - protein, 801 aa, Halalkalibacter krulwichiae
MMKQSKMYGLMIFTFLLLPFLCLQAVTTVGAAEQPSPLLRYENLQLSTGTSKDLSEYVDELSDLDEGTIIVRFRYSGSSFMSLFSLSNNTLPDSHFHLYISPGTIGSENRYQGPDFPKSNIHTRTSSVSLAENYVHTLALVVDKNQGYKYFLNGELVHSDQQSKIAFLDNIHSPNSAKLGKTERSSGNEYLFNGNIDFAEVYSTPLDDQYLLEITGQTHHESLENPLPDDAFITEPSSIFYPGFMDSNNYRIPALYYTMNGTLLAGIDRRVNHGGDSPNDIHAAVRRSFDQGETWETDGIIINAYPDQASNIDLAFTQDETNERIFALVDGFPNGAGLMGGFGNNAYKGTGFKEIDGNSYMFLVDQDENEYTIREEGVVYNQHNEATNYRVDERRDLYLDDEKIDNIFSATTPLTPYKTSYLELYFSDDEGETWTGPIDLNPETKEEWMIFLGVGPGNGIQLKEGTHKGRIVFPVYFLNDHNRQASAVVYSDDNGKTWHRGESPNEGRLLPNGETIREKDFTNHSHEITEAQVVEIPNGQLKMFMRNYSGFAQIATSFDGGETWHEEVVTEEALVAPYSQMSAIRFNGQIDGKEAIIFSSANHPNNRVNGTVRVGLIEEDGTYSNGETNYSIDWRYEQLVKEGHYGYSSLANLESGRIGLFYEATANTNMDYIQFNTEFLKWDRFGDAPKPSIESVQLISQGAISPGQPIQLEVTMDEFVILTGTRSLFGTLAGHQLEFTFVDQLGNQRFLFEATAPQLPPRSYSLTLSVPESLLLYNVYGNKLEDLEPIQSFNKKIAIRK

SEQ ID NO: 5 - protein, 804 aa, Alkalibacillus haloalkaliphilus
MMLKGLLPTLFLTALFLLFTSPGAFAEEIEEEKEPILHVENEEISGGYFTDLNHKIDELKELDQGTIIVRFRHEGSSFQSLFSLSNNNFPNGHFHLYVSPSAIGSENRYQAPGEPQTNTHISQSLTLENDYVHTLAMVVDQEEGYKYYLNGELVLEDTTSSVQFLNNIYEPNSAQLGRTERAAGGNQYTFHGDIDFATVYGEPLQEDYLVELTGETARESLENPLPDDAMVTDPYNVFYPGFMDSNNYRIPALFYTEKGTLIAGIDRRVESGADSPNDIHSAIRRSYDQGETWEDEGIIINAYPDDASNIDLAFTQDNSNERIFALVDGFPHGAGLMGGFGNNAYPGTGYTTVNGEDYMFLLDEDENEYTIREEGIVYDGEGNPTSYKVDENRDLYLDGEKIDNIFSETTPLKPYKTSYLELYHSDDEGDNWTGPIDLNPETKEEWMIFLGVGPGNGIQLSEGEHEGRIIFPVYFINENLRQASAVVYSDDGGETWQRGESPNQGRVVDGNVLDERYFDSPNHEITEAQVVEMPDGQLKMFMRNYSGYAQVATSYDGGETWDSEVITETDLVAPYSQMSAIRYDGQIDGKDAVIFSSANDHTSRINGTVRVGLIEENGPLENGFTDYSFDWKYEQLVKEGHYGYSSLTNLPDGELGLFYEGTPNTVMDFIKFNTDFLKWERHIEEPTPELINYTHLKQEPNVYRKGDSVKVELEFDDFVVLMGDQTLKGEIDGHAIKFNLVESNNSSFTFEATLPDLKPRAYDLNLEFSSDLTIYNKYGKLLDTVYLHFDSSEKIKVTAGNHNE

SEQ ID NO: 6 - protein, 811 aa, Shouchella shacheensis
MTTKLFKFLFVMTMALLIVLQVGEDSPTFAKTKEPVLRVENQAIVDNQFVNLEDEVGQLKELDEGTIIVRFRHTGSSFMSLISLSNKNFPDSHFHFYISPSGVGSENRYEAPGEPKENIHVQSDSLTLQEGEVYTVAMVMDKGEGYKYFLDGELIKEDTQSPRKFLSNVYEPNSAYLGQTDRSQGNSYPFNGDIDFAEVHSEPLSDGVLEDITGETAKSDPVTNPMPDDAFVSEPESIFYPGFMDSPNFRIPALYYTENGTLLAGIDRRVGGGGDSPNDIHAAVRRSLDQGDTWEDDGILINAYPDDASNIDLSFVQDQSSERVFALVDGWPAGAGLMGGFGTNTYKGTGFETIDGKDYMFLTDEDDNRYTIREEGKVYDEGGNSTDYSVDTERNLFQNGEEIDNIFSQTSPLKPFKTSYLELYYSDDEGETWTGPIDLNDQTKEEYMMFLGVGPGNGIQLNEGSNKGRLVFPVYFINDNLRQASAVIYSDDNGETWHRGESPNEGRDVGDGEIINEEDFTNQAHEITEAQVVEMPDGQLKMFMRNYSGYAQIATSFDGGETWDTEIVTETDLVAPYSQMSAIRYDGQVDGQEAVVFSSANHPTSRIDGTVKIGLIEEDGEHKNGYTKYSFDWKYEQLVKQGHYGYSSLTNLPDGEIGLLYEGTANTEMDFMKFNTDFVKWDRVEERPQPELESMNVISSPPPVHRSGEKIQIEATFDDYVMLIGDKRLVGTIAGEEVHFDLVDRTGQDHFVFEAEVPELPPRTHQLEAEFDSDLTIYNLYGKRFSEETKLADTIRVRATGRGGNTFSMSN